(19) United States
(12) Patent Application Publication
(10) Pub. No.: US 2026/0090767 A1
(43) Pub. Date: Apr. 2, 2026

(54) LIVING BODY INFORMATION MEASUREMENT APPARATUS

(71) Applicant: SEIKO EPSON CORPORATION, Tokyo (JP)

(72) Inventors: Tetsuya YAMAMOTO, SUWA-SHI (JP); Yusuke TAKAHASHI, MATSUMOTO-SHI (JP); Osamu YAMADA, CHINO-SHI (JP); Yuya OZAWA, AZUMINO-SHI (JP)

(73) Assignee: SEIKO EPSON CORPORATION, Tokyo (JP)

(21) Appl. No.: 19/344,394

(22) Filed: Sep. 29, 2025

(30) Foreign Application Priority Data
Sep. 30, 2024 (JP) 2024-170173

(51) Int. Cl. A61B 5/00

(52) U.S. Cl. CPC A61B 5/6843; A61B 5/0059; A61B 5/681

(57) ABSTRACT

A living body information measurement apparatus includes a living body information detection apparatus and a contact detector configured to detect contact with a target object. The living body information detection apparatus includes a first light emitting member configured to emit first light toward the target object, a first light receiving member configured to receive the first light from the target object, and a controller configured to acquire living body information on the target object based on the first light received by the first light receiving member. The contact detector is provided in the living body information detection apparatus on a detector side where the first light emitting member and the first light receiving member are provided. The controller is configured to determine a state of contact with the target object based on a value detected by the contact detector.

The present application is based on, and claims priority from JP Application Serial Number 2024-170173, filed September 30, 2024, the disclosure of which is hereby incorporated by reference herein in its entirety.

### BACKGROUND

### 1. Technical Field

The present disclosure relates to a living body information measurement apparatus that is attached to a body and optically detects living body information.

### 2. Related Art

There has been a known living body information measurement apparatus that acquires living body information by using light from a living body sensor unit, acquires a body motion with a body motion sensor, and performs living body evaluation based on the living body information and the body motion (JP-A-2005-253865).

JP-A-2005-253865 is an example of the related art.

In the living body information measurement apparatus described in JP-A-2005-253865 described above, since an acceleration sensor used as the body motion sensor is influenced by the temperature such as the body temperature of a person under measurement, the detected acceleration fluctuates due to the temperature, so that it is difficult to accurately detect the body motion of the person under measurement. It is therefore difficult to determine the contact state resulting from the motion of the body between the living body information measurement apparatus and the living body.

### SUMMARY

A living body information measurement apparatus according to an aspect of the present disclosure includes a living body information detection apparatus and a contact detector configured to detect contact with a target object. The living body information detection apparatus includes a first light emitting member configured to emit first light toward the target object, a first light receiving member configured to receive the first light from the target object, and a controller configured to acquire living body information on the target object based on the first light received by the first light receiving member. The contact detector is provided in the living body information detection apparatus on a detector side where the first light emitting member and the first light receiving member are provided. The controller is configured to determine a state of contact with the target object based on a value detected by the contact detector.

### BRIEF DESCRIPTION OF THE DRAWINGS

FIG. 1 is a bottom view of a living body information measurement apparatus according to a first embodiment.

FIG. 2 is a side cross-sectional view of the living body information measurement apparatus according to the first embodiment.

FIG. 3 is an enlarged bottom view of a sensor apparatus.

FIG. 4 is an enlarged side sectional view of the sensor apparatus.

FIG. 5 is a bottom view showing an example of the state of contact between a target object and a lens member.

FIG. 6 is a bottom view showing an example of the state of contact between the target object and the lens member.

FIG. 7 is a bottom view showing an example of the state of contact between a target object and a lens member.

FIG. 8 is a cross-sectional view showing an example of the state of contact between the target object and the lens member.

FIG. 9 shows an example of the light orientation distribution of first light from a first light emitting member.

FIG. 10 shows an example of the light reception distribution of the first light at a first light receiving member.

FIG. 11 illustrates the arrangement of contact detection members and a contact detection state.

FIG. 12 illustrates the arrangement of the contact detection members and the contact detection state.

FIG. 13 is a bottom view of a living body information measurement apparatus according to a second embodiment.

FIG. 14 is a side cross-sectional view of the living body information measurement apparatus according to the second embodiment.

FIG. 15 is a bottom view showing an example of the state of contact between the target object and the lens member.

FIG. 16 is a bottom view showing an example of the state of contact between the target object and the lens member.

FIG. 17 illustrates a living body information measurement apparatus according to a third embodiment.

FIG. 18 illustrates the living body information measurement apparatus according to the third embodiment.

FIG. 19 illustrates a living body information measurement apparatus according to a fourth embodiment.

FIG. 20 illustrates the living body information measurement apparatus according to the fourth embodiment.

FIG. 21 illustrates the living body information measurement apparatus according to the fourth embodiment.

### DESCRIPTION OF EMBODIMENTS

### First embodiment

A living body information measurement apparatus according to a first embodiment of the present disclosure will be described below with reference to the drawings.

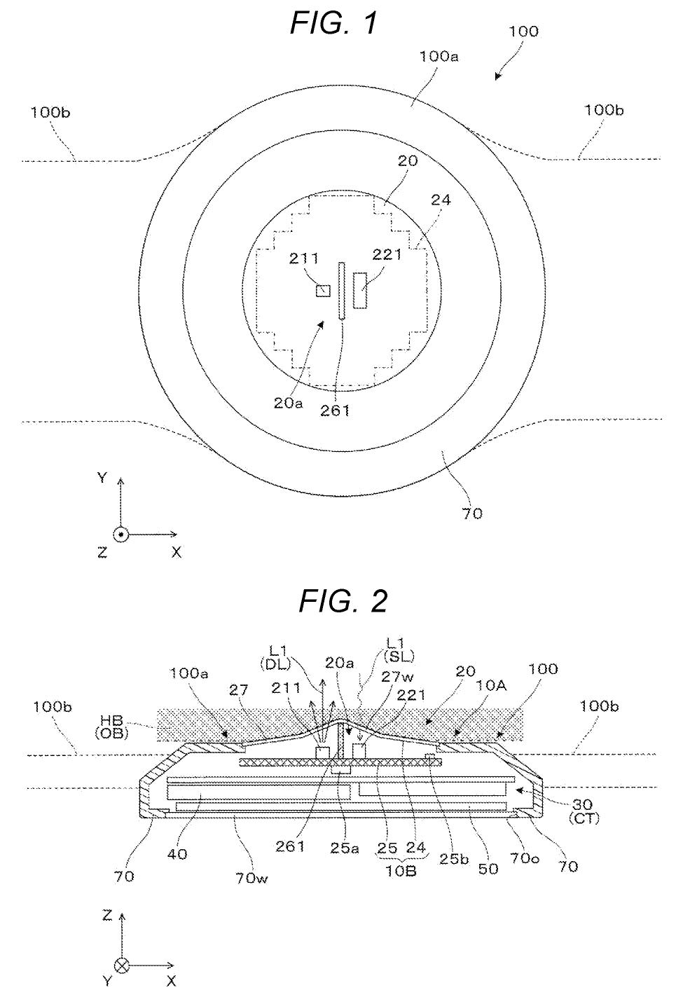

FIG. 1 is a conceptual bottom view illustrating the living body information measurement apparatus according to the first embodiment, and FIG. 2 is a conceptual side cross-sectional view illustrating the living body information measurement apparatus according to the first embodiment.

A living body information measurement apparatus 100 has a wristwatch-shaped appearance, can be worn on an arm that is a portion of a human body, that is, a living body HB, and includes a body apparatus 100a and a pair of wristbands 100b attached to the body apparatus 100a and extending in opposite directions. The body apparatus 100a includes a sensor apparatus 20, a control circuit apparatus 30, a battery 40, a display 50, and an enclosure 70.

In the body apparatus 100a, the sensor apparatus 20 includes a first light emitting member 211, a first light receiving member 221, a contact detector 24, a circuit substrate 25, a partition wall member 261, and a lens member 27. The first light emitting member 211, the first light receiving member 221, the circuit substrate 25, the partition wall member 261, and the lens member 27 function as a living body information detection apparatus 10A. The contact detector 24 and the circuit substrate 25 function as a contact detection apparatus 10B. The first light emitting member 211 is a light emitting diode that emits irradiation light DL, for example, green light, red light, or infrared light. The first light emitting member 211 emits first light L1 that is the irradiation light DL toward the living body HB via a window member 27w of the lens member 27. The first light receiving member 221 is a photodiode that receives return light SL, for example, the green light, red light, or infrared light. The first light receiving member 221 receives the first light L1 that is the return light SL having returned via the window member 27w, and outputs a signal corresponding to the intensity of the first light L1. The combination of the first light emitting member 211 and the first light receiving member 221 is referred to as an optical sensor or a light detector 20a. The light detector 20a can detect pulsation of a blood flow as a fluctuation of the intensity of the first light L1 that is the return light SL, for example, from the artery, and can acquire information on the pulse rate or the like.

The contact detector 24 is provided on a side of the circuit substrate 25 that is the side where the light detector 20a, which is provided with the first light emitting member 211 and the first light receiving member 221, is provided, and detects contact with the living body HB, which is a target object OB. Specifically, the contact detector 24 is provided at the lens member 27. The contact detector 24 is, for example, a current sensor using electrostatic induction, and detects a current generated by contact with the living body HB. In the present specification, a description stating that the contact detector 24 is in a contact state, that is, is in contact with the living body HB or the target object OB also indicates, as a result of the living body HB or the target object OB coming into contact with the lens member 27, that the target object OB is brought into the vicinity of the contact detector 24 and is in indirect contact with the contact detector 24.

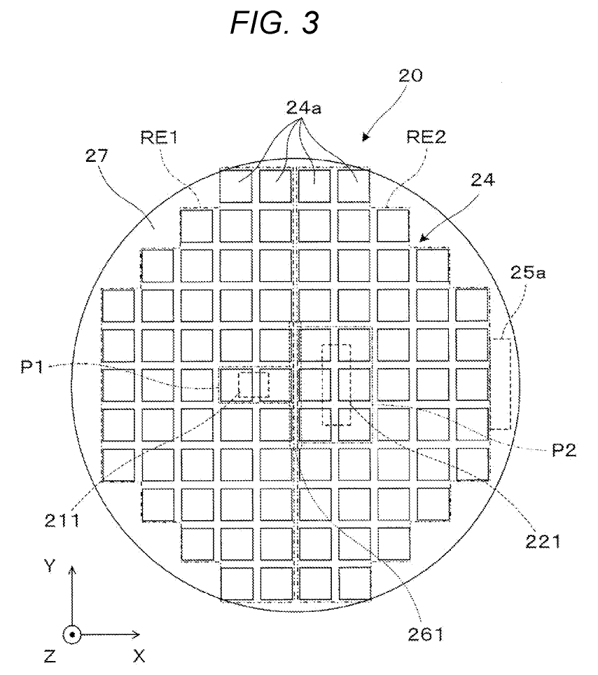

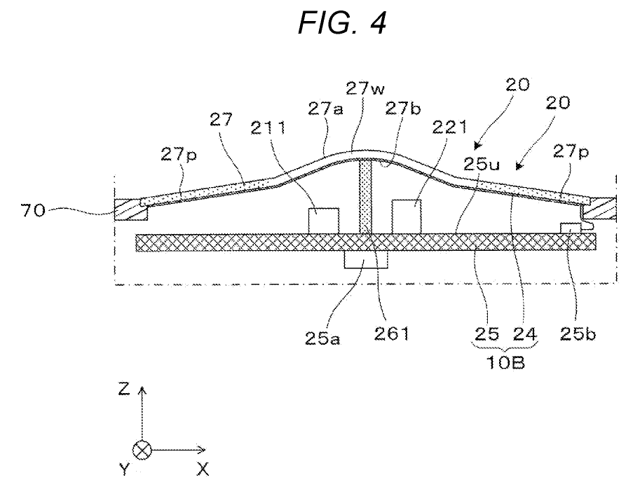

FIGS. 3 and 4 are enlarged bottom and side cross-sectional views of the sensor apparatus 20. The contact detector 24 includes multiple contact detection members 24a two-dimensionally arranged in a matrix. The contact detection members 24a are each a rectangular transparent electrode made, for example, of ITO, and are attached and fixed to an inner surface 27b of the lens member 27. The contact detection members 24a each generate a weak electrostatically induced current when the target object OB, that is, the living body HB comes into contact with a local region of an outer surface 27a of the lens member 27 that is a region corresponding to the contact detection members 24a, or the vicinity of the local region. Although not shown, wires independent from each other extend from the contact detection members 24a, and are coupled to a second drive circuit 25b. A detection signal according to whether there is contact is output from each of the contact detection members 24a to the second drive circuit 25b. The detection signals from the contact detector 24 allow determination of the state of contact between the target object OB and the lens member 27, specifically, an increase or decrease in the contact region or a change in bias of the contact region. The state of contact between the target object OB and the lens member 27 reflects a motion of the target object OB on which the living body information measurement apparatus 100 is worn, and corresponds to information on a motion of the living body HB (body motion).

Although not shown, the contact detection members 24a may not necessarily be formed at the inner surface 27b of the lens member 27 but may be formed at the outer surface 27a thereof.

Each of the contact detection members 24a is not limited to a transparent electrode and may, for example, be a rectangular electrically conductive frame. In this case, the four sides of the rectangular shape are configured with thin metal strips. The rectangular electrically conductive frame itself blocks the first light L1, but the opening of the electrically conductive frame transmits the first light L1. The contact detection members 24a that are each an electrically conductive frame each generate a weak electrostatically induced current when the target object OB, that is, the living body HB comes into contact with a local region of the outer surface 27a of the lens member 27 that is a region corresponding to the electrically conductive frame that forms the contact detection members 24a, or the vicinity of the local region.

In the contact detector 24 or the lens member 27, a first region RE1 has a first portion P1, which overlaps with the first light emitting member 211 in the plan view or the bottom view, and a second region RE2 has a second portion P2, which overlaps with the first light receiving member 221 in the plan view or the bottom view. In the first region RE1 or the first portion P1, when the target object OB is not in contact with the lens member 27, the intensity of the first light L1 incident on the target object OB from the first light emitting member 211 may be relatively weaker than that in the case where they are in contact with each other. In the second region RE2 or the second portion P2, when the target object OB is not in contact with the lens member 27, the intensity of the first light L1 incident on the first light receiving member 221 from the target object OB may be relatively weaker than that in the case where they are in contact with each other. The decrease in the intensity of the first light L1 resulting from the contact state described above can be compensated for by correction of increasing the intensity of the first light L1 emitted from the first light emitting member 211 or increasing the sensitivity at which the first light L1 is detected by the first light receiving member 221.

The circuit substrate 25 supports the first light emitting member 211, the first light receiving member 221, and the partition wall member 261 on an upper surface 25u and fixes the members thereto. The circuit substrate 25 includes a first drive circuit 25a, which operates the first light emitting member 211 and the first light receiving member 221, and the second drive circuit 25b, which operates the contact detector 24. The first drive circuit 25a outputs an optical response from the target object OB, which is the living body HB, specifically, information on the pulse to the control circuit apparatus 30. Information on the state of contact between the target object OB and the lens member 27 is output from the second drive circuit 25b to the control circuit apparatus 30.

The partition wall member 261 separates a space in which the first light emitting member 211 is disposed and a space in which the first light receiving member 221 is disposed from each other. Specifically, the partition wall member 261 is provided between the first light emitting member 211 and the first light receiving member 221 in the ±X direction, in which the first light emitting member 211 and the first light receiving member 221 are arranged, and extends in the ±Y direction perpendicular to the direction in which the first light emitting member 211 and the first light receiving member 221 are arranged. The partition wall member 261 extends to a height close to the lens member 27 in the vertical +Z direction. The partition wall member 261 is made, for example, of a black resin material, has low thermal conductivity, and blocks light. The partition wall member 261 prevents the first light L1 emitted from the first light emitting member 211 from directly entering the first light receiving member 221. The partition wall member 261 further prevents radiant heat from the first light emitting member 211 from being transmitted to the first light receiving member 221. Note that the material of a body of the circuit substrate 25 is also made of a material that has low thermal conductivity and blocks light. As a result, the space in which the first light emitting member 211 is disposed and the space in which the first light receiving member 221 is disposed are optically open only in the direction toward the window member 27w of the lens member 27.

The lens member 27 includes the window member 27w, which is a central portion of the lens member 27, and an outer frame member 27p, which is the periphery thereof, and is disposed so as to overlap with the first light emitting member 211 and the first light receiving member 221 in the plan view or the bottom view in the +Z direction. The lens member 27 is made, for example, of a material such as glass or resin, and is so light transmissive that it efficiently transmits the first light L1, and is so insulating that it prevents the target object OB and the contact detector 24 from being electrically conductive to each other.

Referring back to FIGS. 1 and 2, the control circuit apparatus 30 is an arithmetic processing circuit including a microprocessor, operates the sensor apparatus 20, measures living body information based on a value detected by the light detector 20a, and measures and determines the state of contact between the target object OB and the lens member 27 and the state of the motion of the target object OB based on a value detected by the contact detector 24. The control circuit apparatus 30 and the circuit substrate 25 are collectively referred to as a controller CT. The control circuit apparatus 30 or the controller CT, for example, acquires living body information such as the pulse of the target object OB based on the first light L1 received by the first light receiving member 221, and acquires body motion information on the state of the motion of the target object OB based on the state of the contact with the target object OB detected by the contact detection members 24a. The control circuit apparatus 30 can also correct the operation of the light detector 20a based on the state of the contact with the target object OB acquired by using the contact detector 24. The control circuit apparatus 30 can cause the display 50 to display a result of the measurement made by the sensor apparatus 20. The control circuit apparatus 30 may include a communication circuit and an antenna that allow digital communication with an external instrument.

The battery 40 supplies electric power to the sensor apparatus 20 and the control circuit apparatus 30 to operate the sensor apparatus 20 and the control circuit apparatus 30. The battery 40 may be a rechargeable secondary battery. In this case, a charging circuit or the like can be incorporated into the enclosure 70 of the living body information measurement apparatus 100.

The display 50 is a liquid crystal panel or an organic EL display, performs a display operation under the control of the control circuit apparatus 30, and displays various pieces of information such as a result of the measurement. The display 50 is disposed in the enclosure 70 on a side of the circuit substrate 25 that is the side opposite the lens member 27, and is covered with a plate-shaped window member 70w, which is light transmissive and fitted into an opening 70o.

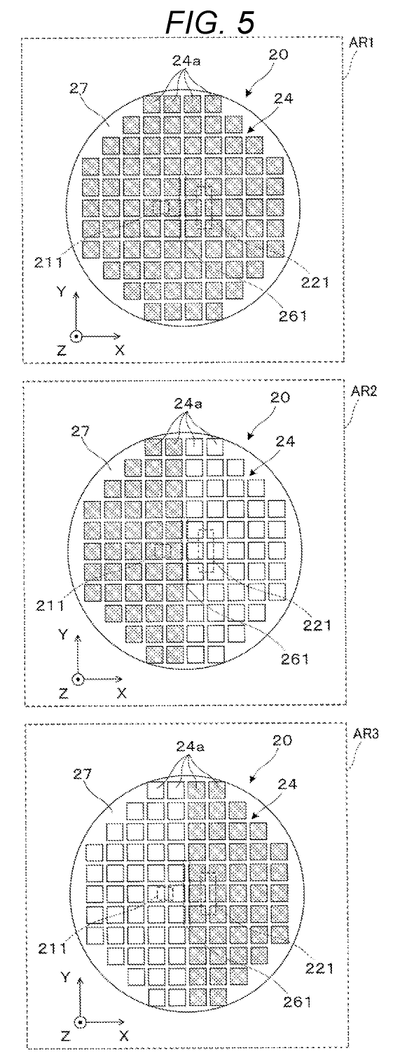

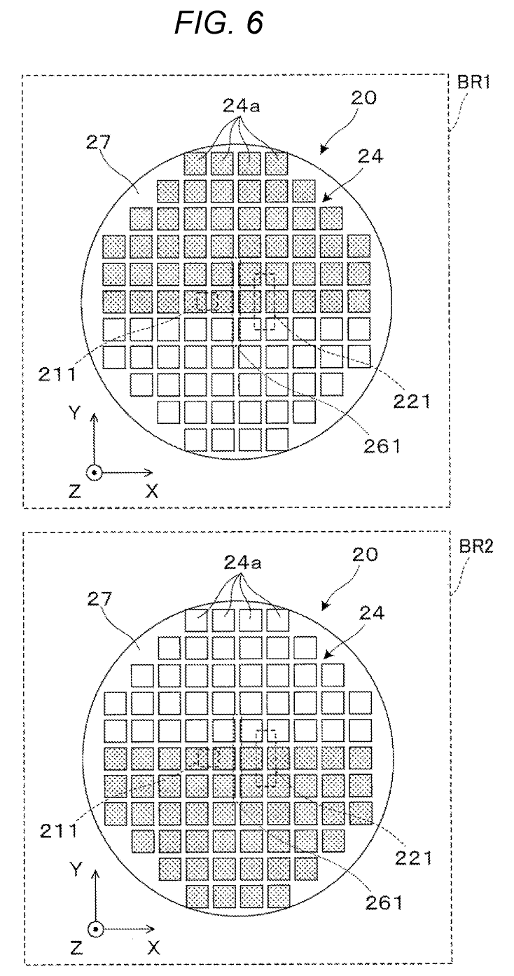

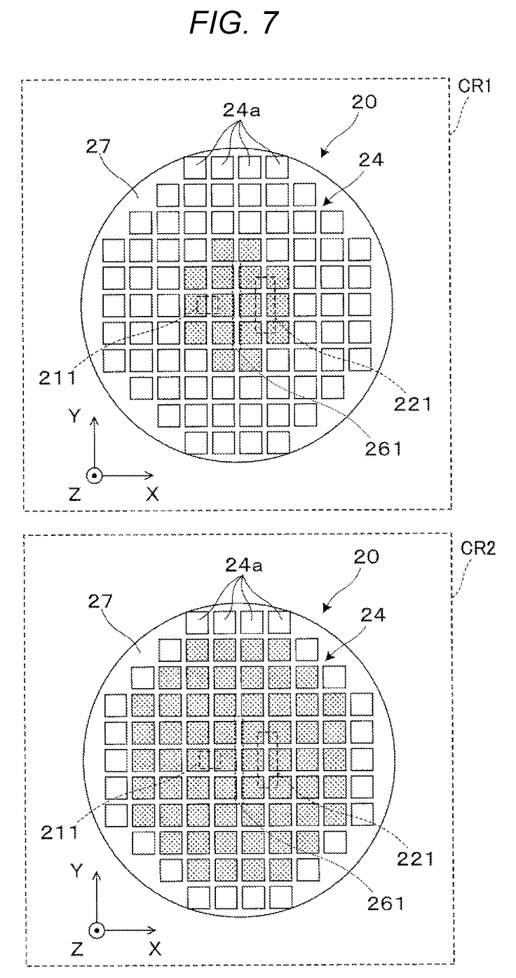

FIGS. 5 to 7 are bottom views illustrating the state of contact between the target object OB and the lens member 27. The contact detection members 24a corresponding to the lens member 27 with which the target object OB is in contact, that is, the contact detection members 24a detecting the contact are hatched in a dot pattern. In FIG. 5, a region AR1 shows a state in which the target object OB is in contact with the entire lens member 27, a region AR2 shows a state in which the target object OB is in contact with the left half (−X side) of the lens member 27, and a region AR3 shows a state in which the target object OB is in contact with the right half (+X side) of the lens member 27. In FIG. 6, a region BR1 shows a state in which the target object OB is in contact with the upper half (+Y side) of the lens member 27, and a region BR2 shows a state in which the target object OB is in contact with the lower half (−Y side) of the lens member 27. In FIG. 7, a region CR1 shows a state in which the target object OB is in contact with a central portion of the lens member 27 in a biased manner, and a region CR2 shows a state in which the target object OB is in contact with substantially the entire lens member 27 excluding the outer edge thereof.

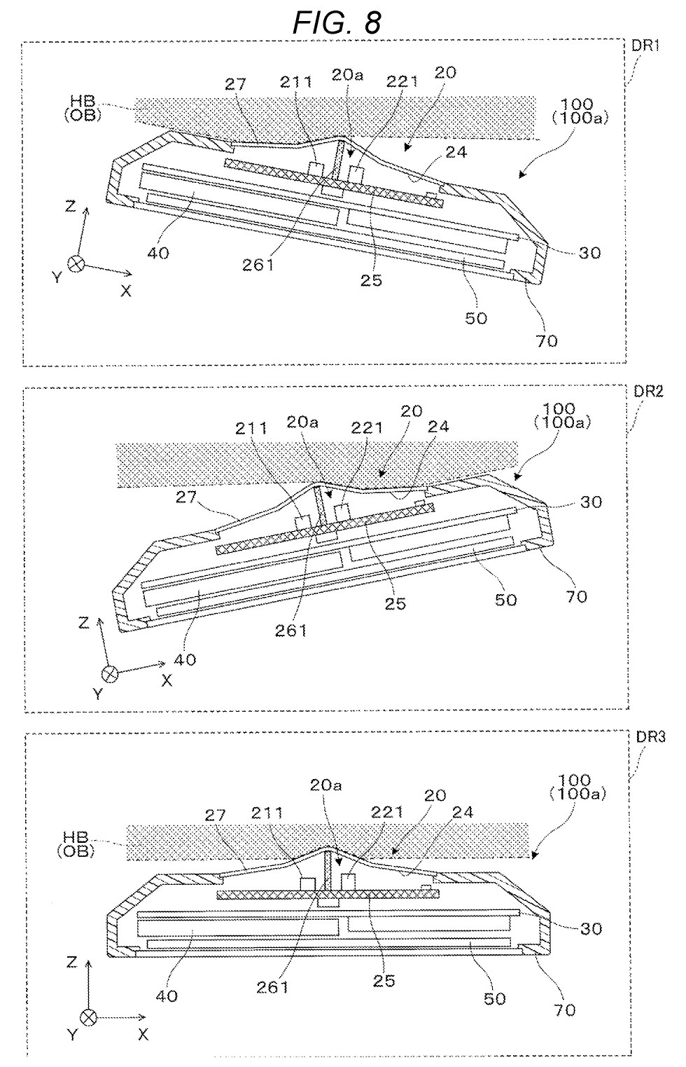

FIG. 8 is a cross-sectional view illustrating the state of contact between the target object OB and the lens member 27. In FIG. 8, a region DR1 shows the state in which the target object OB is in contact with the left half (−X side) of the lens member 27, and corresponds to the state shown in the region AR2 in FIG. 5. In FIG. 8, a region DR2 shows the state in which the target object OB is in contact with the right half (+X side) of the lens member 27, and corresponds to the state shown in the region AR3 in FIG. 5. In FIG. 8, a region DR3 shows the state in which the target object OB is in contact with a central portion of the lens member 27 in a biased manner, and corresponds to the state shown in the region CR1 in FIG. 7.

For example, when the fluctuation of the contact region as shown in the regions AR2 and AR3 in FIG. 5 is repeated, it is conceivable that the target object OB makes a pivotal reciprocating motion, that is, swinging motion around the Y-axis with respect to the sensor apparatus 20. When the fluctuation of the contact region as shown in the regions BR1 and BR2 in FIG. 6 is repeated, it is conceivable that the target object OB makes a pivotal reciprocating motion, that is, swinging motion around the X-axis with respect to the sensor apparatus 20. When the fluctuation of the contact region as shown in the regions CR1 and CR2 in FIG. 7 is repeated, it is conceivable that the target object OB makes a reciprocating translating motion in the Z-axis direction with respect to the sensor apparatus 20. The fluctuation of the contact region shown in FIGS. 5 and 6 can be determined from a positional fluctuation of the contour of the region of the contact detection members 24a indicating contact (hereinafter, also referred to as contact region) or the center of gravity of the contact region. The control circuit apparatus 30 performs data processing on such a positional fluctuation of the contact region in the form of velocity or acceleration in the X direction or the Y direction. The fluctuation of the contact region shown in FIG. 7 can be determined from an increase or a decrease in the area or the contour length of the contact region of the contact detection members 24a. The control circuit apparatus 30 performs data processing on such an increase or a decrease in the contact region in the form of velocity or acceleration in the Z direction. As described above, information on the fluctuation of the contact region of the contact detector 24 in the X direction, the Y direction, and the Z direction is managed as body motion information reflecting a three-dimensional motion of the target object OB or the living body HB in the control circuit apparatus 30.

In the case of the inclination shown in the region DR2 in FIG. 8, the contact region of the contact detection members 24a is biased to the right half, and the first light L1 that is the irradiation light DL from the first light emitting member 211 is incident via an air layer on the skin of the target object OB separate from the lens member 27. It is estimated that the intensity of the first light L1 incident on the target object OB present farther away from the lens member 27 is relatively lower than the intensity of the first light L1 incident on the target object OB in close contact with the lens member 27, and it is believed that the lower intensity influences the result of measurement of a blood flow or the like. To cancel or reduce the influence, it is desirable to increase the intensity of the light emitted from the first light emitting member 211.

When the first light L1 from the first light emitting member 211 is incident on the skin of the target object OB partly separate from the lens member 27, it is more desirable to consider the light orientation distribution of the first light L1 emitted from the first light emitting member 211.

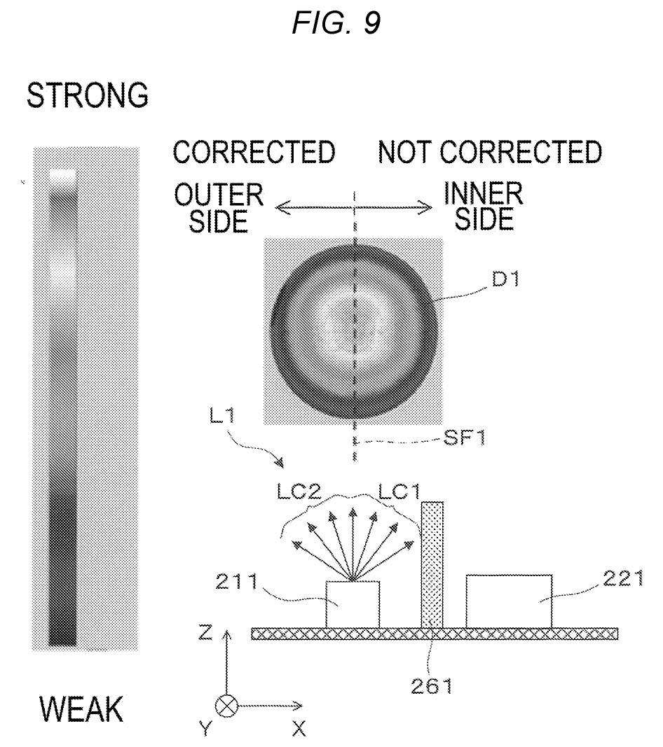

FIG. 9 shows an example of a light orientation distribution D1 of the first light L1 from the first light emitting member 211. Out of the first light L1 emitted from the first light emitting member 211, components LC1 passing through the center of the first light emitting member 211 and traveling inward, that is, toward the right side (+X side) of a reference plane SF1 parallel to the YZ direction, are highly likely to enter the first light receiving member 221 via the target object OB, while components LC2 traveling outward, that is, toward the left side (−X side) of the reference plane SF1, are less likely to enter the first light receiving member 221 via the target object OB. Therefore, when the sensor apparatus 20 inclines with respect to the target object OB, and the skin of the target object OB is separate from the lens member 27 in the region outside (−X side of) the reference plane SF1, it is believed that an appropriate measurement result can be rather acquired by not making correction in which the intensity of the light emitted from the first light emitting member 211 is increased.

Conversely, in the case of the inclination shown in the region DR1 in FIG. 8, the contact region of the contact detection members 24a is biased to the left half of the lens member 27, and the first light L1 that is the return light SL from the skin of the target object OB separate from the lens member 27 enters the first light receiving member 221 via the air layer. It is estimated that the intensity of the first light L1 entering the first light receiving member 221 from the target object OB present farther away from the lens member 27 is relatively lower than the intensity of the first light L1 entering the first light receiving member 221 from the target object OB in close contact with the lens member 27, and it is believed that the lower intensity influences the result of measurement of a blood flow or the like. To cancel or reduce the influence, it is desirable to increase the detection sensitivity of the first light receiving member 221.

When the first light L1 output from the skin of the target object OB partly separate from the lens member 27 enters the first light receiving member 221, it is more desirable to consider a light reception distribution D2 at the first light receiving member 221.

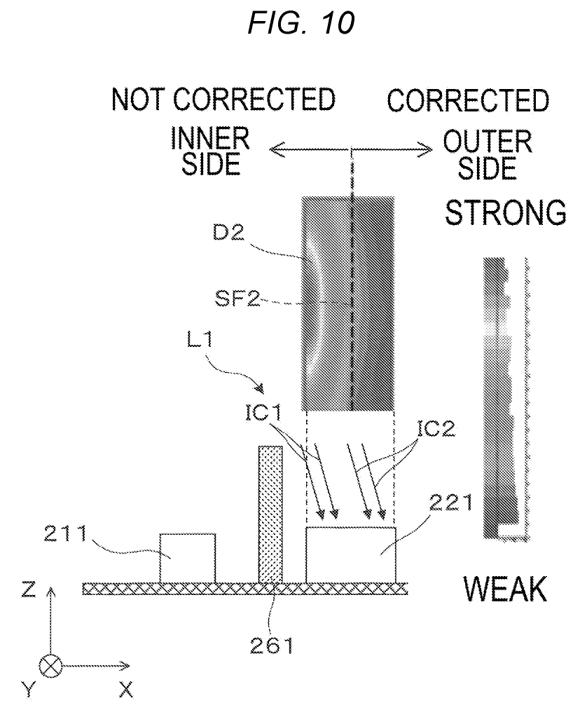

FIG. 10 shows the light reception distribution D2 of the first light L1 at the first light receiving member 221. Out of the first light L1 entering the first light receiving member 221, components IC1 passing through the center of the first light receiving member 221 and incident on an inner side, which is the left side (−X side) of a reference plane SF2 parallel to the YZ direction, have a relatively high intensity, while components IC2 incident on an outer side, which is the right side (+X side) of the reference plane SF2, have a relatively low intensity. That is, at the first light receiving member 221, the distribution of the return light SL from the skin of the target object OB is concentrated on the side where the first light emitting member 211 is disposed. Therefore, when the sensor apparatus 20 inclines with respect to the target object OB, and the skin of the target object OB is separate from the lens member 27 in the region outside (+X side of) the reference plane SF2, it is believed that an appropriate measurement result can be rather acquired by not making correction in which the detection sensitivity of the first light receiving member 221 is increased.

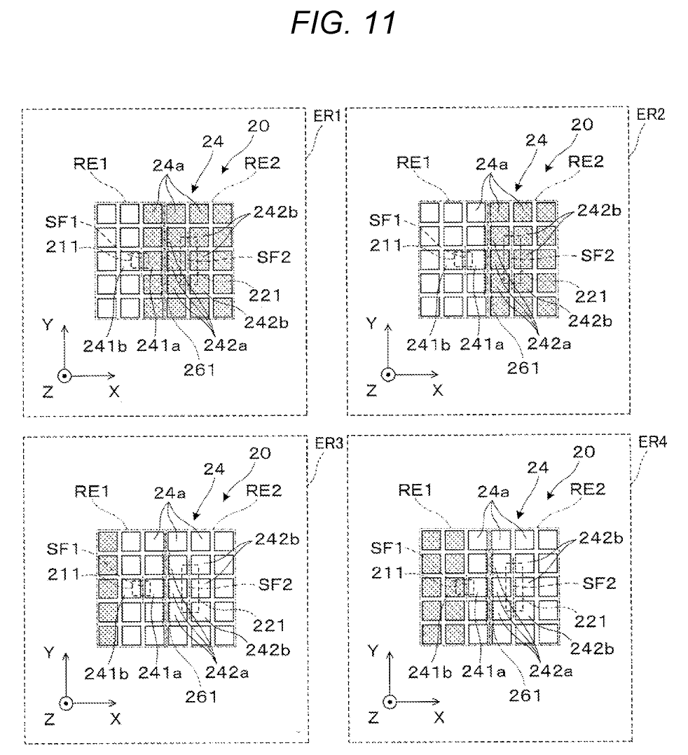

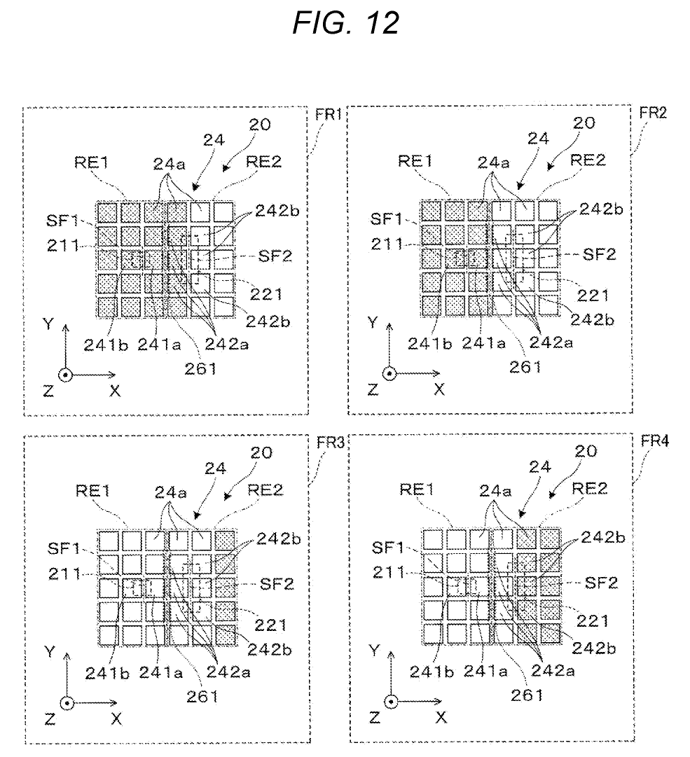

FIGS. 11 and 12 illustrate the arrangement of the contact detection members 24a, which constitute the contact detector 24, with respect to the first light emitting member 211 and the first light receiving member 221, and a state in which contact is detected by the contact detection members 24a. In FIG. 11, regions ER1, ER2, ER3, and ER4 show specific examples in which the contact state changes primarily on the side where the first light emitting member 211 is disposed. In FIG. 12, regions FR1, FR2, FR3, and FR4 show specific examples in which the contact state changes primarily on the side where the first light receiving member 221 is disposed.

Referring to the region ER1 and the like shown in FIG. 11, the contact detection members 24a, which constitute the contact detector 24, are provided in the first region RE1 facing the first light emitting member 211 and the second region RE2 facing the first light receiving member 221.

In the contact detector 24 or the lens member 27, the first region RE1 includes first portions 241a and 241b (corresponding to first portion P1 in FIG. 3), which overlap with the first light emitting member 211 in the plan view or the bottom view. In the example shown in FIG. 11, one element 241a of the first portions 241a and 241b is located on the +X side of the reference plane SF1 and close to the first light receiving member 221, and the other element 241b of the first portions 241a and 241b is located on the −X side of the reference plane SF1 and far from the first light receiving member 221.

In the contact detector 24 or the lens member 27, the second region RE2 includes second portions 242a and 242b (corresponding to second portion P2 in FIG. 3), which overlap with the first light receiving member 221 in the plan view or the bottom view. In the example shown in FIG. 11, three elements 242a of the second portions 242a and 242b are located on the −X side of the reference plane SF2 and close to the first light emitting member 211, and the other three elements 242b of the second portions 242a and 242b are located on the +X side of the reference plane SF2 and far from the first light emitting member 211.

In the state shown in the region ER1 in FIG. 11, the control circuit apparatus 30, which is the controller CT shown in FIG. 2, determines that the element 241b of the first portions 241a and 241b, which is farther from the second portions 242a and 242b, is in a non-contact state and the element 241a of the first portions 241a and 241b, which is closer to the second portions 242a and 242b, is in a contact state. In this case, the control circuit apparatus 30 does not correct the intensity of the first light L1 emitted by the first light emitting member 211. When the intensity of the first light L1 reaching the first light receiving member 221 is unlikely to be influenced on the side where the first light emitting member 211 is disposed, the correction is not made, so that the processing load can be reduced.

In the state shown in the region ER2 in FIG. 11, the control circuit apparatus 30, which is the controller CT, determines that the element 241a of the first portions 241a and 241b, which is on the side close to the second portions 242a and 242b, and the element 241b of the first portions 241a and 241b, which is on the side far from the second portions 242a and 242b, are both in the non-contact state. In this case, the control circuit apparatus 30 corrects the intensity of the first light L1 emitted by the first light emitting member 211 to be higher than the intensity of the first light L1 emitted by the first light emitting member 211 in the case where the control circuit apparatus 30 determines that at least the element 241a closer to the second portions 242a and 242b out of the first portions 241a and 241b is in the contact state. Correction in accordance with the contact states and the light radiation distribution can thus be made.

In the state shown in the region ER3 in FIG. 11, the control circuit apparatus 30, which is the controller CT, determines that the element 241b of the first portions 241a and 241b, which is on the side far from the second portions 242a and 242b, and the element 241a of the first portions 241a and 241b, which is on the side close to the second portions 242a and 242b, are both in the non-contact state. In this case, the control circuit apparatus 30 corrects the intensity of the first light L1 emitted by the first light emitting member 211 to be higher than the intensity of the first light L1 emitted by the first light emitting member 211 in the case where the control circuit apparatus 30 determines that at least the element 241a closer to the second portions 242a and 242b out of the first portions 241a and 241b is in the contact state. Note in this case that the control circuit apparatus 30 determines that the second portions 242a and 242b, which overlap with the first light receiving member 221 in the plan view, are in the non-contact state, and makes correction in which the detection sensitivity or the light reception sensitivity of the first light receiving member 221 is increased. The correction will be described later in detail.

In the state shown in the region ER4 in FIG. 11, the control circuit apparatus 30, which is the controller CT, determines that the element 241b on the side farther from the second portions 242a and 242b out of the first portions 241a and 241b is in the contact state, and the element 241a on the side closer to the second portions 242a and 242b out of the first portions 241a and 241b is in the non-contact state. In this case, the control circuit apparatus 30 corrects the intensity of the first light L1 emitted by the first light emitting member 211 to be higher than the intensity of the first light L1 emitted by the first light emitting member 211 in the case where the control circuit apparatus 30 determines that the element 241b on the far side is in the non-contact state and the element 241a on the close side is in the contact state.

In the state shown in the region FR1 of FIG. 12, the control circuit apparatus 30, which is the controller CT, determines that the element 242b of the second portions 242a and 242b, which is farther from the first portions 241a and 241b, is in the non-contact state, and the element 242a of the second portions 242a and 242b, which is closer to the first portions 241a and 241b, is in the contact state. In this case, the control circuit apparatus 30 does not correct the light reception sensitivity at which the first light receiving member 221 receives the first light L1. When the intensity of the received first light L1 is unlikely to be influenced on the side where the first light receiving member 221 is disposed, the correction is not made, so that the processing load can be reduced.

In the state shown in the region FR2 in FIG. 12, the control circuit apparatus 30, which is the controller CT, determines that the element 242a of the second portions 242a and 242b, which is on the side closer to the first portions 241a and 241b, and the element 242b of the second portions 242a and 242b, which is on the side farther from the first portions 241a and 241b, are both in the non-contact state. In this case, the control circuit apparatus 30 corrects the light reception sensitivity at which the first light receiving member 221 receives the first light L1 to be higher than the light reception sensitivity at which the first light receiving member 221 receives the first light L1 in the case where the control circuit apparatus 30 determines that at least the element 242a on the side closer to the first portions 241a and 241b out of the second portions 242a and 242b is in the contact state. Correction in accordance with the contact states and the light radiation distribution can thus be made.

In the state shown in the region FR3 in FIG. 12, the control circuit apparatus 30, which is the controller CT, determines that the element 242b of the second portions 242a and 242b, which is on the side farther from the first portions 241a and 241b, and the element 242a of the second portions 242a and 242b, which is on the side closer to the first portions 241a and 241b, are both in the non-contact state. In this case, the control circuit apparatus 30 corrects the light reception sensitivity at which the first light receiving member 221 receives the first light L1 to be higher than the light reception sensitivity at which the first light receiving member 221 receives the first light L1 in the case where the control circuit apparatus 30 determines that at least the element 242a on the side closer to the first portions 241a and 241b out of the second portions 242a and 242b is in the contact state.

In the state shown in the region FR4 in FIG. 12, the control circuit apparatus 30, which is the controller CT, determines that the element 242b of the second portions 242a and 242b, which is on the side farther from the first portions 241a and 241b, is in the contact state, and the element 242a of the second portions 242a and 242b, which is on the side closer to the first portions 241a and 241b, is in the non-contact state. In this case, the control circuit apparatus 30 corrects the light reception sensitivity at which the first light receiving member 221 receives the first light L1 to be higher than the light reception sensitivity at which the first light receiving member 221 receives the first light L1 in the case where the control circuit apparatus 30 determines that the element 242b on the farther side is in the non-contact state and the element 242a on the closer side is in the contact state.

As a result, in the state shown in the region ER1 in FIG. 11 and the state shown in the region FR1 in FIG. 12, the central portion that overlaps with the first light emitting member 211 and the first light receiving member 221 out of the contact detector 24 is in the contact state, so that the correction is not made, as in the inclination shown in the region DR3 in FIG. 8.

In the operation described above, the control circuit apparatus 30, which is the controller CT, controls the first light emitting member 211 and the first light receiving member 221 in accordance with the contact states of the first region RE1, that is, the first portions 241a and 241b, and the second region RE2, that is, the second portions 242a and 242b. The control described above allows the intensity of the light emitted from the first light emitting member 211 to be corrected in accordance with the contact state on the side where the first light emitting member 211 is disposed, or the light reception sensitivity of the first light receiving member 221 to be corrected in accordance with the contact state on the side where the first light receiving member 221 is disposed, so that the accuracy of the detection made by the living body information measurement apparatus 100 can be improved.

The size, arrangement, and density of the contact detection members 24a shown in FIGS. 11 and 12 are merely examples. For example, it is not necessary to arrange a boundary in the contact detection members 24a in such a way that the contact detection members 24a sandwich the center of the first light emitting member 211 or the reference plane SF1, and it is not necessary to arrange a boundary in the contact detection members 24a in such a way that the contact detection members 24a sandwich the center of the first light receiving member 221 or the reference plane SF2.

The correction of the intensity of the light emitted from the first light emitting member 211 or the light reception sensitivity of the first light receiving member 221 can be set as appropriate based on the arrangement of the contact detection members 24a with respect to the first light emitting member 211 and the first light receiving member 221, the contact state of the contact detection members 24a, and other factors. For example, when a boundary between a group of contact detection members 24a in the contact state and a group of contact detection members 24a in the non-contact state is present at a position separate outward from the front surface of the first light emitting member 211, the amount of correction of the intensity of the emitted light can be relatively increased in accordance with the distance from the front surface to the boundary. When a boundary between a group of contact detection members 24a in the contact state and a group of contact detection members 24a in the non-contact state is present at a position separate outward from the front surface of the first light receiving member 221, the amount of correction of the light reception sensitivity can be relatively increased in accordance with the distance from the front surface to the boundary.

The living body information detected by the light detector 20a is influenced by a fluctuation of the state of contact between the target object OB or the living body HB and the lens member 27 or the contact detector 24, in particular, a fluctuation in the inclination from the Z-axis toward the X direction or the Y direction as shown in FIG. 8. The control circuit apparatus 30 may therefore carry out the process of removing a disturbance-induced noise component from a living body signal detected by the light detector 20a by using the signal detected by the contact detector 24 and relating to the body motion. For example, assuming that the living body signal acquired by the light detector 20a has a frequency of 60 Hz, and that the body motion signal acquired by the contact detector 24 has a frequency of 10 Hz or 90 Hz, the accuracy of the living body signal is improved by removing a noise component having the frequency of 10 Hz or 90 Hz from the living body signal acquired by the light detector 20a. That is, the control circuit apparatus 30 or the controller CT determines the noise component contained in the signal according to the first light L1 received by the first light receiving member 221 based on a change in the contact states of the first region RE1 and the second region RE2, and acquires living body information on the target object OB from the signal which is derived from the first light L1 but from which the noise component has been removed. The noise component applied to the living body information measurement apparatus 100 can thus be determined and removed in accordance with the contact states.

As described above, the living body information measurement apparatus 100 according to the first embodiment includes the living body information detection apparatus 10A and the contact detector 24, which detects contact with the target object OB, the living body information detection apparatus 10A includes the first light emitting member 211, which emits the first light L1 toward the target object OB, the first light receiving member 221, which receives the first light L1 from the target object OB, and the controller CT, which acquires living body information on the target object OB based on the first light L1 received by the first light receiving member 221, the contact detector 24 is provided in the living body information detection apparatus 10A on the detector side where the first light emitting member 211 and the first light receiving member 221 are provided, and the controller CT determines the state of contact with the target object OB based on a value detected by the contact detector 24.

In the living body information measurement apparatus 100 described above, since the contact detector 24 provided on the detector side in the living body information detection apparatus 10A detects the contact with the target object OB, and the controller CT determines the state of the contact with the target object OB based on a value detected by the contact detector 24, the contact state resulting from a motion of the living body HB or the body of the person under measurement, which is the target object OB, can be determined without use of an acceleration sensor influenced by the temperature such as the body temperature of the person under measurement.

### Second embodiment

A living body information measurement apparatus according to a second embodiment will be described below. Note that the living body information measurement apparatus according to the second embodiment is a partly changed version of the living body information measurement apparatus according to the first embodiment, and portions common to those of the living body information measurement apparatus according to the first embodiment will not be described.

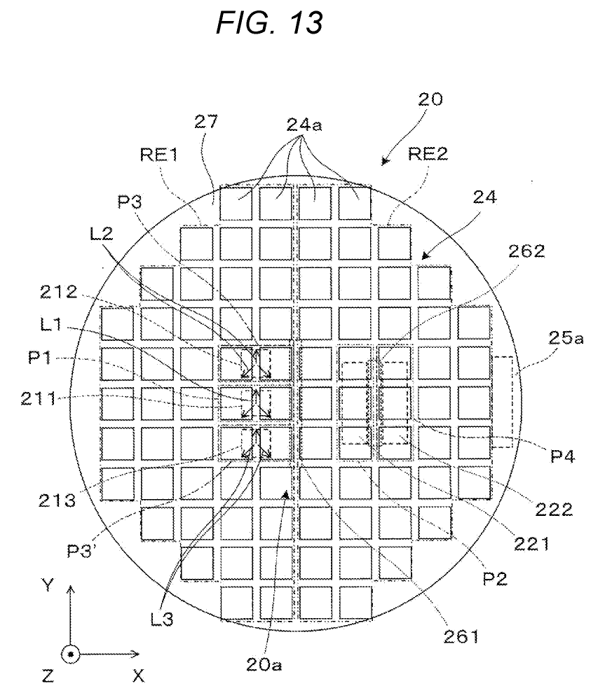

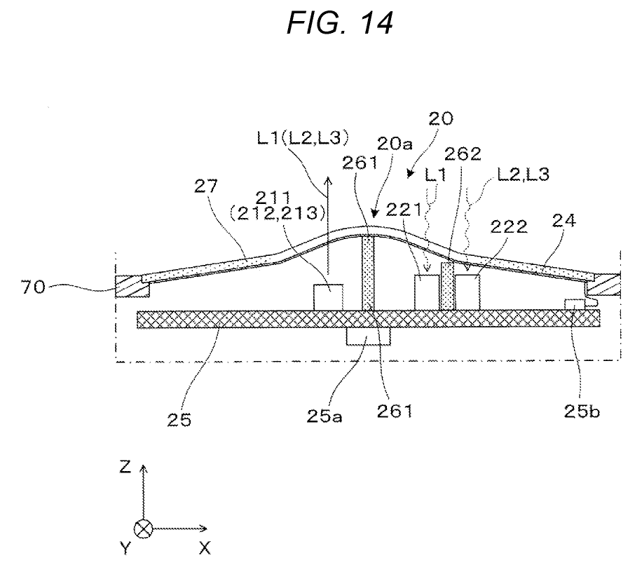

FIG. 13 is a bottom view illustrating the living body information measurement apparatus according to the second embodiment, and FIG. 14 is a side cross-sectional view illustrating the living body information measurement apparatus according to the second embodiment. The living body information measurement apparatus 100 according to the second embodiment includes the first light emitting member 211, a pair of second light emitting members 212 and 213, the first light receiving member 221, and a second light receiving member 222 as the light detector 20a.

The first light emitting member 211 is a light emitting diode that emits green light as the first light L1, the second light emitting member 212, which is one of the pair of second light emitting members 212 and 213, is a light emitting diode that emits red light having a longer wavelength than that of the first light L1 as second light L2, and the second light emitting member 213, which is the other one of the pair of second light emitting members 212 and 213, is a light emitting diode that emits infrared light having a longer wavelength than that of the second light L2 as third light L3. The first light receiving member 221 is a photodiode that receives the first light L1 that is green return light and outputs a signal corresponding to the intensity of the first light L1, and the second light receiving member 222 is a photodiode that receives the second light L2 that is red return light and the third light L3 that is infrared return light and outputs signals corresponding to the intensities of the second light L2 and the third light L3.

In the contact detector 24 or the lens member 27, the first region RE1 includes the first portion P1, which overlaps with the first light emitting member 211 in the plan view or the bottom view, and third portions P3 and P3', which overlap with the second light emitting members 212 and 213 in the plan view or the bottom view, and the second region RE2 includes the second portion P2, which overlaps with the first light receiving member 221 in the plan view or the bottom view, and a fourth portion P4, which overlaps with the second light receiving member 222 in the plan view or the bottom view.

A partition wall member 262, which separates the first light receiving member 221 and the second light receiving member 222 from each other, is disposed therebetween.

The first light emitting member 211 and the second light emitting members 212 and 213 can be operated at different timings, and the second light receiving member 222 can detect the ratio between the intensity of the second light L2 that is the red return light and the intensity of the third light L3 that is the infrared return light. The measurement using the combination of the green first light L1, the red second light L2, and the infrared third light L3 allows not only measurement of the pulse rate but also measurement of the in-blood oxygen saturation.

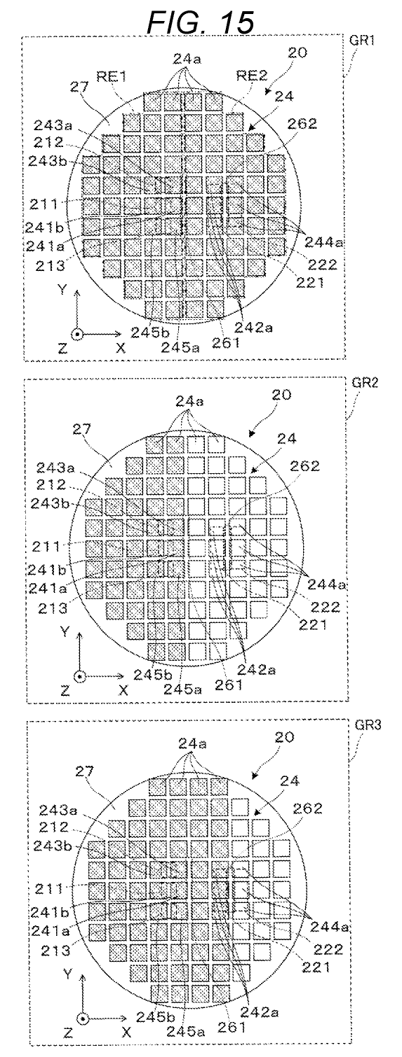

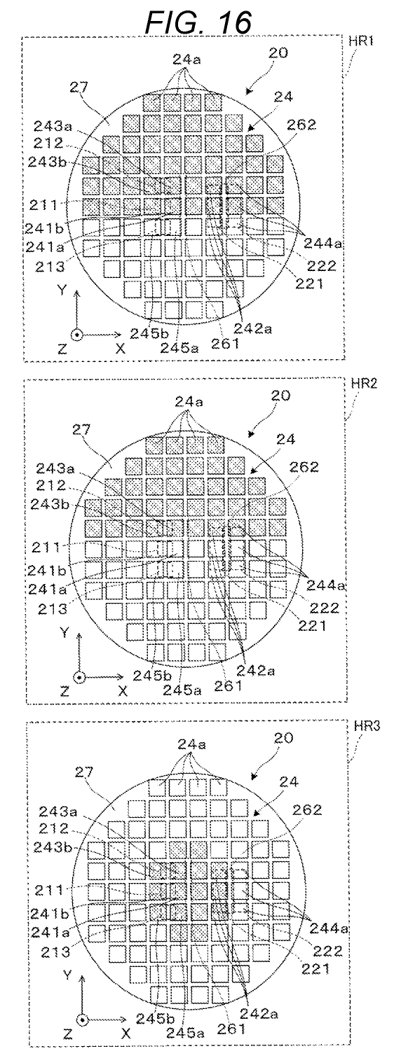

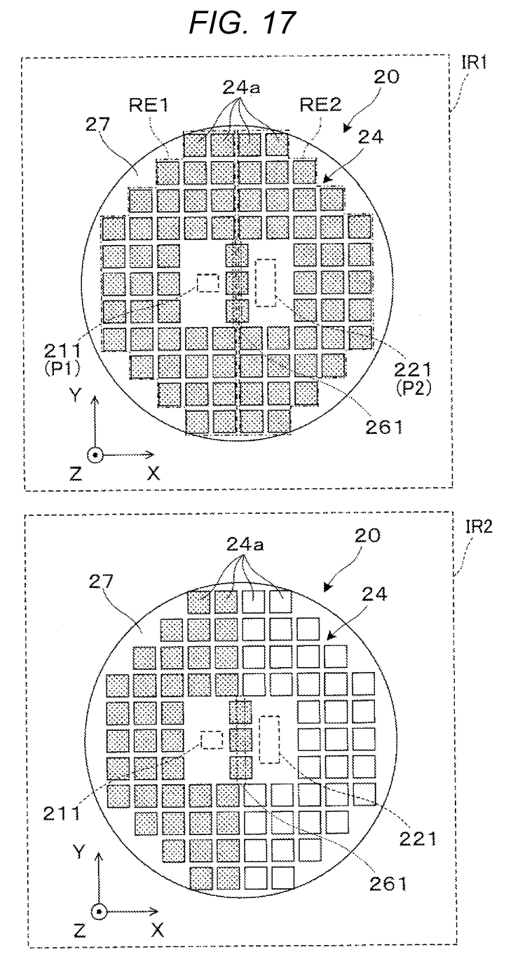

FIGS. 15 and 16 are bottom views illustrating the state of contact between the target object OB and the lens member 27 or the contact detector 24. The contact detection members 24a corresponding to the lens member 27 with which the target object OB is in contact, that is, the contact detection members 24a detecting the contact are hatched in a dot pattern. In FIG. 15, a region GR1 shows a state in which the target object OB is in contact with the entire lens member 27, a region GR2 shows a state in which the target object OB is in contact with the left half (−X side) of the lens member 27, and a region GR3 shows a state in which the target object OB is in contact with the left half (−X side) of the lens member 27 and a portion that extends rightward off the left half. In FIG. 16, a region HR1 shows a state in which the target object OB is in contact with a portion slightly wider than the upper half (+Y side) of the lens member 27, a region HR2 shows a state in which the target object OB is in contact with a portion slightly narrower than the upper half (+Y side) of the lens member 27, and a region HR3 shows a state in which the target object OB is in contact with a central portion of the lens member 27 in a biased manner.

Referring to the region GR1 shown in FIG. 15, the contact detection members 24a, which constitute the contact detector 24, are provided in the first region RE1 facing the first light emitting member 211 and the second region RE2 facing the first light receiving member 221.

In the contact detector 24 or the lens member 27, the first region RE1 includes the first portions 241a and 241b (corresponding to first portion P1 in FIG. 13), which overlap with the first light emitting member 211, which emits the green light, in the plan view or the bottom view, third portions 243a and 243b (corresponding to third portion P3 in FIG. 13), which overlap with the second light emitting member 212, which emits the red light, in the plan view or the bottom view, and third portions 245a and 245b (corresponding to third portion P3' in FIG. 13), which overlap with the second light emitting member 213, which emits the infrared light, in the plan view or the bottom view. In the contact detector 24 or the lens member 27, the second region RE2 includes the second portions 242a (corresponding to second portion P2 in FIG. 13), which overlap with the first light receiving member 221, which receives the green light, in the plan view or the bottom view, and includes fourth portions 244a (corresponding to fourth portion P4 in FIG. 13), which overlap with the second light receiving member 222, which receives the red light and the infrared light, in the plan view or the bottom view.

In the state shown in the region GR2 in FIG. 15, the control circuit apparatus 30, which is the controller CT shown in FIG. 2, determines that the second portions 242a of the second portions 242a and the fourth portions 244a, which are closer to the first portions 241a and 241b, and the fourth portions 244a of the second portions 242a and the fourth portions 244a, which are farther from the first portions 241a and 241b, are both in the non-contact state. In this case, the control circuit apparatus 30 corrects the light reception sensitivity at which the first light receiving member 221 receives the first light L1 to be higher than the light reception sensitivity at which the first light receiving member 221 receives the first light L1 in the case where the control circuit apparatus 30 determines that the second portions 242a are in the contact state. The control circuit apparatus 30 further corrects the light reception sensitivity at which the second light receiving member 222 receives the second light L2 and the third light L3 to be higher than the light reception sensitivity at which the second light receiving member 222 receives the second light L2 and the third light L3 in the case where the control circuit apparatus 30 determines that the fourth portions 244a are in the contact state.

In the state shown in the region GR3 in FIG. 15, the control circuit apparatus 30 determines that the second portions 242a of the second portions 242a and the fourth portions 244a, which are closer to the first portions 241a and 241b, are in the contact state, and the fourth portions 244a of the second portions 242a and the fourth portions 244a, which are farther from the first portions 241a and 241b, are in the non-contact state. In this case, the control circuit apparatus 30 does not correct the light reception sensitivity at which the first light receiving member 221 receives the first light L1. On the other hand, the control circuit apparatus 30 corrects the light reception sensitivity at which the second light receiving member 222 receives the second light L2 and the third light L3 to be higher than the light reception sensitivity at which the second light receiving member 222 receives the second light L2 and the third light L3 in the case where the control circuit apparatus 30 determines that the fourth portions 244a are in the contact state.

In the state shown in the region HR1 in FIG. 16, the control circuit apparatus 30 determines that the first portions 241a and 241b are in the contact state, and does not correct the intensity of the first light L1 emitted by the first light emitting member 211. The control circuit apparatus 30 determines that the third portions 243a and 243b are in the contact state, and does not correct the intensity of the second light L2 emitted by the second light emitting member 212. On the other hand, the control circuit apparatus 30 determines that the third portions 245a and 245b are in the non-contact state, and corrects the intensity of the third light L3 emitted by the second light emitting member 213 to be higher than the intensity of the third light L3 emitted by the second light emitting member 213 in the case where the control circuit apparatus 30 determines that the element 245a on the side closer to the second portions 242a is in the contact state.

In the state shown in the region HR1 in FIG. 16, the control circuit apparatus 30 determines that some of the second portions 242a and the fourth portions 244a are in the non-contact state on the −Y side or the lower side. In this case, the control circuit apparatus 30 corrects the light reception sensitivity at which the first light receiving member 221 receives the first light L1 to be higher in accordance with the proportion of the non-contact state than the light reception sensitivity at which the first light receiving member 221 receives the first light L1 in the case where the control circuit apparatus 30 determines that all the second portions 242a are in the contact state. The control circuit apparatus 30 further corrects the light reception sensitivity at which the second light receiving member 222 receives the second light L2 and the third light L3 to be higher in accordance with the proportion of the non-contact state than the light reception sensitivity at which the second light receiving member 222 receives the second light L2 and the third light L3 in the case where the control circuit apparatus 30 determines that all the fourth portions 244a are in the contact state.

In the state shown in the region HR2 in FIG. 16, the control circuit apparatus 30 determines that the third portions 243a and 243b are in the contact state, and does not correct the intensity of the second light L2 emitted by the second light emitting member 212. On the other hand, the control circuit apparatus 30 determines that the first portions 241a and 241b are in the non-contact state, and corrects the intensity of the first light L1 emitted by the first light emitting member 211 to be higher than the intensity of the first light L1 emitted by the first light emitting member 211 in the case where the control circuit apparatus 30 determines that the element 241a on the side closer to the second portions 242a is in the contact state. The control circuit apparatus 30 further determines that the third portions 245a and 245b are in the non-contact state, and corrects the intensity of the third light L3 emitted by the second light emitting member 213 to be higher than the intensity of the third light L3 emitted by the second light emitting member 213 in the case where the control circuit apparatus 30 determines that the element 245a on the side closer to the second portions 242a is in the contact state.

In the state shown in the region HR2 in FIG. 16, the control circuit apparatus 30 determines that some of the second portions 242a and the fourth portions 244a are in the contact state on the +Y side or the upper side. In this case, the control circuit apparatus 30 corrects the light reception sensitivity at which the first light receiving member 221 receives the first light L1 to be higher in accordance with the proportion of the non-contact state than the light reception sensitivity at which the first light receiving member 221 receives the first light L1 in the case where the control circuit apparatus 30 determines that all the second portions 242a are in the contact state. The control circuit apparatus 30 further corrects the light reception sensitivity at which the second light receiving member 222 receives the second light L2 and the third light L3 to be higher in accordance with the proportion of the non-contact state than the light reception sensitivity at which the second light receiving member 222 receives the second light L2 and the third light L3 in the case where the control circuit apparatus 30 determines that all the fourth portions 244a are in the contact state.

In the state shown in the region HR3 in FIG. 16, the control circuit apparatus 30 shows a state in which the lens member 27 and the target object OB are in contact with each other at a central portion of the lens member 27 in a biased manner. In this case, the control circuit apparatus 30 does not correct the light reception sensitivity at which the first light receiving member 221 receives the first light L1, but corrects the light reception sensitivity at which the second light receiving member 222 receives the second light L2 and the third light L3, as in the state shown in the region GR3 in FIG. 15.

In the example of the arrangement shown in FIG. 13, the position and the width of the second portion P2 substantially coincide with the position and the width of the first light receiving member 221 in the X direction, and the position and the width of the fourth portion P4 substantially coincide with the position and the width of the second light receiving member 222 in the X direction, but the second portion P2 and the fourth portion P4 on the light reception side may also be provided across multiple contact detection members 24a in the X direction, as the first portion P1 and the like on the light emission side.

### Third embodiment

A living body information measurement apparatus according to a third embodiment will be described below. Note that the living body information measurement apparatus according to the third embodiment is a partly changed version of the living body information measurement apparatus according to the first embodiment.

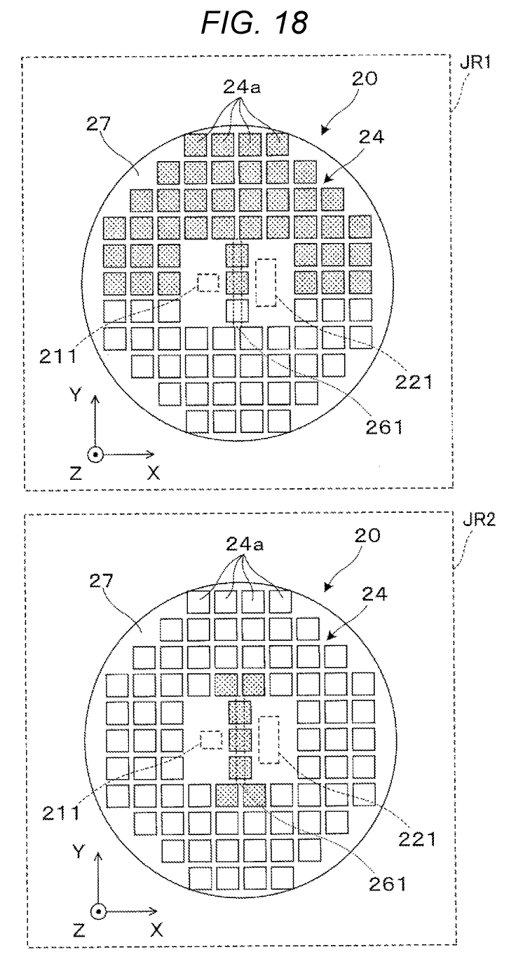

FIGS. 17 and 18 are bottom views illustrating the living body information measurement apparatus according to the third embodiment. In FIG. 17, a region IR1 shows a state in which the target object OB is in contact with the entire lens member 27, and a region IR2 shows a state in which the target object OB is in contact with the left half (-X side) of the lens member 27. In FIG. 18, a region JR1 shows a state in which the target object OB is in contact with the upper half (+Y side) of the lens member 27, and a region JR2 shows a state in which the target object OB is in contact with a central portion of the lens member 27 in a biased manner.

In the third embodiment, in the contact detector 24 or the lens member 27, the contact detection members 24a are not provided in the first portion P1 of the first region RE1. The contact detection members 24a are each configured with a transparent electrode or the like, but tend to lower the transmittance for the first light L1. Providing no contact detection members 24a in the first portion P1 can achieve a situation in which the contact detection members 24a do not block the first light L1 from the first light emitting member 211. Furthermore, in the third embodiment, in the contact detector 24 or the lens member 27, the contact detection members 24a are not provided in the second portion P2 of the second region RE2. The configuration described above can achieve a situation in which the contact detection members 24a do not block the first light L1 entering the first light receiving member 221.

Also in the third embodiment, the body motion information can be measured by the contact detector 24. Note that although the contact state in each of the first portion P1 and the second portion P2 cannot be directly detected, the intensity of the first light L1 emitted by the first light emitting member 211 can be corrected, and the sensitivity at which the first light receiving member 221 receives the first light L1 can be corrected based on the state of the contact detection members 24a disposed around the first portion P1 and the second portion P2.

### Fourth embodiment

A living body information measurement apparatus according to a fourth embodiment will be described below. Note that the living body information measurement apparatus according to the fourth embodiment is a partly changed version of the living body information measurement apparatus according to the first or second embodiment.

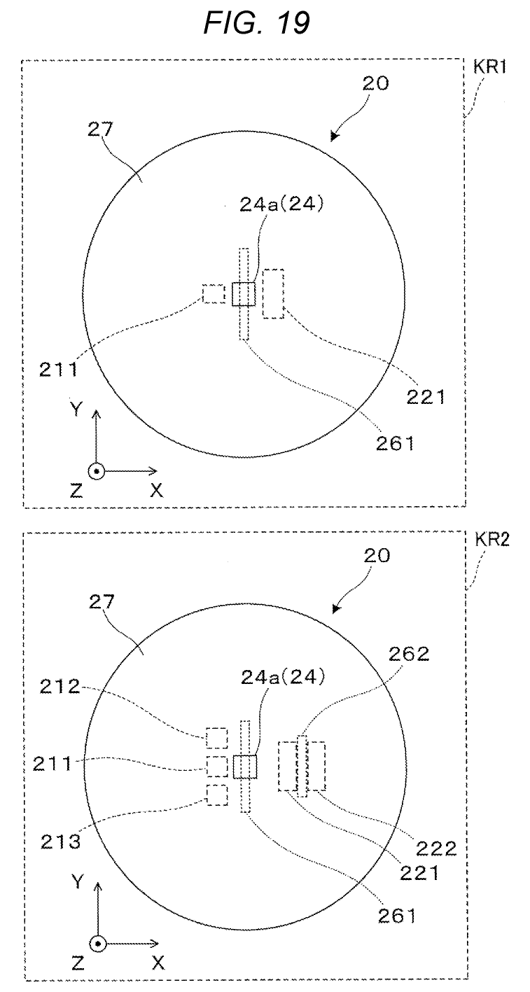

FIG. 19 is a bottom view illustrating the living body information measurement apparatus according to the fourth embodiment. In FIG. 19, a region KR1 shows the arrangement of the contact detector 24 used in the light detector 20a of the type shown in FIG. 3, which illustrates the first embodiment. In FIG. 19, a region KR2 shows the arrangement of the contact detector 24 used in the light detector 20a of the type shown in FIG. 13, which illustrates the second embodiment.

In the case of the contact detector 24 shown in FIG. 19, a single contact detection member 24a is provided at the center of the lens member 27. In this case, the structure of the contact detector 24 can be simplified. However, whether contact with the single contact detection member 24a has occurred is only detected, but the direction of a body motion of the target object OB cannot be identified.

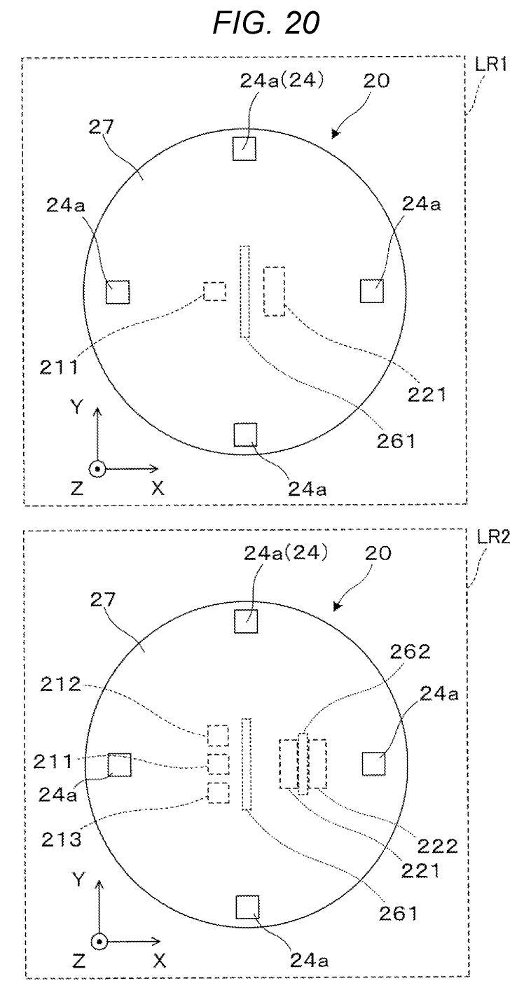

FIG. 20 is a bottom view illustrating a living body information measurement apparatus according to a variation. In FIG. 20, a region LR1 shows the arrangement of the contact detector 24 used in the light detector 20a of the type shown in FIG. 3, which illustrates the first embodiment, and a region LR2 shows the arrangement of the contact detector 24 used in the light detector 20a of the type shown in FIG. 13, which illustrates the second embodiment.

In the case of the contact detector 24 shown in FIG. 20, four contact detection members 24a are provided at four positions arranged at equal angular intervals at the periphery of the lens member 27. In this case, the contact detector 24, although having a simple configuration, can detect the inclination of the light detector 20a with respect to the ±X and ±Y directions, so that the direction of a body motion of the target object OB can be identified.

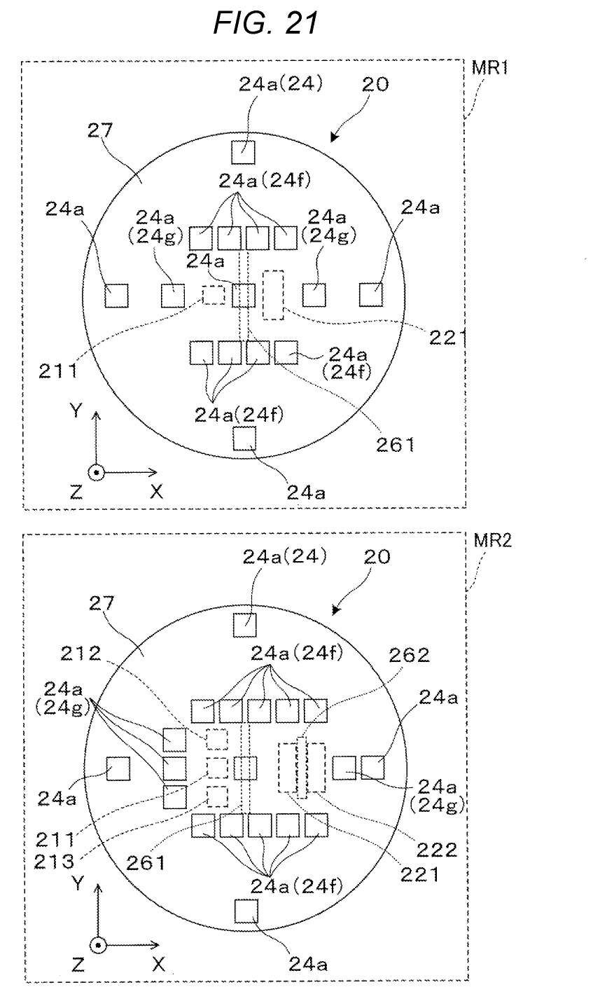

FIG. 21 is a bottom view illustrating a living body information measurement apparatus according to another variation. In FIG. 21, a region MR1 shows the arrangement of the contact detector 24 used in the light detector 20a of the type shown in FIG. 3, which illustrates the first embodiment, and a region MR2 shows the arrangement of the contact detector 24 used in the light detector 20a of the type shown in FIG. 13, which illustrates the second embodiment.

In the case of the contact detector 24 shown in the region MR1 in FIG. 21, the contact detection members 24a are provided at the center of the lens member 27, the periphery of the lens member 27, and positions along a ring surrounding the first light emitting member 211 and the first light receiving member 221, which constitute the light detector 20a. In particular, contact detection members 24f can be used to detect the contact state of the contact detector 24 resulting from rotation around the Y-axis or inclination with respect to the X direction, and can be used to determine the necessity of correction comparable to the correction described with reference to FIGS. 11 and 12. Note that contact detection members 24g can be used to determine whether portions outside the first light emitting member 211 and the first light receiving member 221 are in the contact state.

Also in the case of the contact detector 24 shown in the region MR2 in FIG. 21, the contact detection members 24a are provided at the center of the lens member 27, the periphery of the lens member 27, and positions along a ring surrounding the first light emitting member 211 and the first light receiving member 221, which constitute the light detector 20a. The functions of the contact detection member 24f and the contact detection member 24g are the same as those of the contact detector 24 shown in the region MR1 in FIG. 21.

### Variations and others

The present disclosure has been described above with reference to the embodiments, but is not limited to the embodiments described above, and can be implemented in various aspects without departing from the key points of the present disclosure. For example, variations below are conceivable.

The living body information measurement apparatus 100 is not limited to a wristwatch-shaped wearable instrument that measures the pulse rate or the like, and can be used as a smartwatch, an activity meter, or the like having various functions.

The living body information measurement apparatus 100 is not limited to an apparatus worn on a wrist, and may be an apparatus that brings the sensor apparatus 20 into close contact with any site of the body.

A living body information measurement apparatus according to a specific aspect includes a living body information detection apparatus and a contact detector configured to detect contact with a target object, the living body information detection apparatus includes a first light emitting member configured to emit first light toward the target object, a first light receiving member configured to receive the first light from the target object, and a controller configured to acquire living body information on the target object based on the first light received by the first light receiving member, the contact detector is provided in the living body information detection apparatus on a detector side where the first light emitting member and the first light receiving member are provided, and the controller is configured to determine a state of contact with the target object based on a value detected by the contact detector.

In the living body information measurement apparatus described above, since the contact detector provided on the detector side in the living body information detection apparatus detects the contact with the target object, and the controller determines the state of the contact with the target object based on a value detected by the contact detector, the state of contact resulting from a motion of a living body or the body of the person under measurement, which is the target object, can be determined without use of an acceleration sensor influenced by the temperature such as the body temperature of the person under measurement.

In a specific aspect, the controller is configured to control the first light receiving member and the first light emitting member in accordance with the determined state of contact. That is, the detection operation of the living body information detection apparatus can be corrected in accordance with the state of contact detected by the contact detector.

In a specific aspect, the contact detector includes a plurality of contact detection members, the plurality of contact detection members each transmit a detection signal to the controller when coming into contact with the target object, and the controller determines that the target object and the living body information measurement apparatus are in contact with each other by receiving the detection signals from the plurality of contact detection members. In this case, the state of contact between the target object and the living body information detection apparatus can be determined at multiple locations based on the signals from the plurality of contact detection members.

In a specific aspect, the living body information measurement apparatus includes a lens member configured to transmit the first light toward the target object and transmit the first light from the target object toward the first light receiving member, and the plurality of contact detection members are provided at the lens member. In this case, the state of contact between the target object and the living body information measurement apparatus can be determined with respect to the lens member.

In a specific aspect, the plurality of contact detection members are provided in a first region having a first portion that overlaps with the first light emitting member and a second region having a second portion that overlaps with the first light receiving member, and the controller is configured to control the first light emitting member and the first light receiving member in accordance with contact states of the first region and the second region. The intensity of the light emitted from the first light emitting member can be corrected in accordance with the contact state of the side where the first light emitting member is disposed, or the light reception sensitivity of the first light receiving member can be corrected in accordance with the contact state of the side where the first light receiving member is disposed, so that the accuracy of the detection made by the living body information detection apparatus can be increased.

In a specific aspect, when the first portion of the first region is in a contact state and at least a part of the second portion of the second region is in a non-contact state, the controller is configured to correct a light reception sensitivity at which the first light receiving member receives the first light to be higher than the light reception sensitivity at which the first light receiving member receives the first light in a case where the second portion of the second region is in the contact state. The light reception sensitivity of the first light receiving member can thus be corrected in accordance with the degree of contact on the side where the first light receiving member is disposed.

In a specific aspect, when the controller determines that the entire second portion of the second region is in the non-contact state, the controller is configured to correct the light reception sensitivity at which the first light receiving member receives the first light to be higher than the light reception sensitivity at which the first light receiving member receives the first light in a case where the controller determines that a part of the second portion of the second region is in the non-contact state. The correction can therefore be so made that the light reception sensitivity in the case where the entire second portion is in a non-contact state is higher than that in the case where a part of the second portion is in a non-contact state.

In a specific aspect, when the controller determines that a side of the second portion of the second region that is closer to the first portion of the first region is in the non-contact state, and a side of the second portion of the second region that is farther from the first portion of the first region is in the contact state, the controller is configured to correct the light reception sensitivity at which the first light receiving member receives the first light to be higher than the light reception sensitivity at which the first light receiving member receives the first light in a case where the controller determines that the farther side is in the non-contact state and the closer side is in the contact state. Correction in accordance with the contact states and the light reception distribution can thus be made.

In a specific aspect, when the controller determines that the side of the second portion of the second region that is farther from the first portion of the first region is in the non-contact state, and the side of the second portion of the second region that is closer to the first portion of the first region is in the contact state, the controller is configured not to correct the light reception sensitivity at which the first light receiving member receives the first light. When the intensity of the received first light is unlikely to be influenced on the side where the first light receiving member is disposed, the correction is not made, so that the processing load can be reduced.

In a specific aspect, when the second portion of the second region is in the contact state and at least a part of the first portion of the first region is in the non-contact state, the controller is configured to correct an intensity of the first light emitted by the first light emitting member to be higher than the intensity of the first light emitted by the first light emitting member in a case where the first portion of the first region is in the contact state. Increasing the intensity of the first light in the case where the side where the first light emitting member is disposed is in the contact state, that is, in the non-contact state in which the first light is unlikely to reach the target object allows the first light to be likely to reach the target object, so that the accuracy of the detection made by the living body information detection apparatus can be increased.

In a specific aspect, when the controller determines that the entire first portion of the first region is in the non-contact state, the controller is configured to correct the intensity of the first light emitted by the first light emitting member to be higher than the intensity of the first light emitted by the first light emitting member in a case where the controller determines that a part of the first portion of the first region is in the non-contact state. The correction can therefore be so made that the intensity of the emitted light in the case where the entire first portion is in a non-contact state is higher than that in the case where a part of the first portion is in a non-contact state.

In a specific aspect, when the controller determines that a side of the first portion of the first region that is closer to the second portion of the second region is in the non-contact state, and a side of the first portion of the first region that is farther from the second portion of the second region is in the contact state, the controller is configured to correct the intensity of the first light emitted by the first light emitting member to be higher than the intensity of the first light emitted by the first light emitting member in a case where the controller determines that the farther side is in the non-contact state and the closer side is in the contact state. Correction in accordance with the contact states and the light radiation distribution can thus be made.

In a specific aspect, when the controller determines that the side of the first portion of the first region that is farther from the second portion of the second region is in the non-contact state, and the side of the first portion of the first region that is closer to the second portion of the second region is in the contact state, the controller is configured not to correct the intensity of the first light emitted by the first light emitting member. When the intensity of the first light reaching the first light receiving member is unlikely to be influenced on the side where the first light emitting member is disposed, the correction is not made, so that the processing load can be reduced.

In a specific aspect, the controller is configured to determine a noise component contained in a signal derived from the first light received by the first light receiving member based on a change in the contact states of the first region and the second region, and acquire living body information on the target object from the signal which is derived from the first light but from which the noise component is removed. The noise component applied to the living body information detection apparatus can thus be determined and removed in accordance with the contact states.

In a specific aspect, at the lens member, the plurality of contact detection members are not provided in the first portion of the first region. The configuration described above can achieve a situation in which the plurality of contact detection members do not block the first light from the first light emitting member.

In a specific aspect, at the lens member, the plurality of contact detection members are not provided in the second portion of the second region. The configuration described above can achieve a situation in which the plurality of contact detection members do not block the first light entering the first light receiving member.

In a specific aspect, the living body information measurement apparatus includes a second light emitting member configured to emit second light having a wavelength longer than a wavelength of the first light, and a second light receiving member configured to receive the second light, the first region of the lens member has a third portion that overlaps with the second light emitting member, the second region of the lens member has a fourth portion that overlaps with the second light receiving member, and the controller is configured to control the second light emitting member and the second light receiving member in accordance with contact states of the third portion and the fourth portion. Even when there are multiple light emitting members and light receiving members, control according to the contact states can be performed.

In a specific aspect, the fourth portion of the second region is farther from the first region than the second portion of the second region, and when the first region is in the contact state and the fourth portion of the second region is in the non-contact state, the controller is configured to correct the light reception sensitivity at which the second light receiving member receives the second light in such a way that the light reception sensitivity increases, and not correct the light reception sensitivity at which the first light receiving member receives the first light. Increasing the light reception sensitivity of the second light receiving member when the second light receiving member far from the side where the light emitter is disposed is in the non-contact state and making no correction of the light reception sensitivity of the first light receiving member that is not greatly influenced allow accurate control of the amount of light received by each of the light receiving members.

In a specific aspect, the controller is configured to correct the intensity of the second light emitted by the second light emitting member in a case where the third portion of the first region is in the non-contact state to be higher than the intensity of the first light emitted by the first light emitting member in a case where the third portion of the first region is in the non-contact state. Increasing the intensity of the second light when the third portion is in the non-contact state, the second light is likely to reach the target object.

What is claimed is:

1. A living body information measurement apparatus, comprising:
a living body information detection apparatus; and
a contact detector configured to detect contact with a target object,
wherein the living body information detection apparatus includes:
a first light emitting member configured to emit first light toward the target object,
a first light receiving member configured to receive the first light from the target object, and
a controller configured to acquire living body information on the target object based on the first light received by the first light receiving member,
the contact detector is provided in the living body information detection apparatus on a detector side where the first light emitting member and the first light receiving member are provided, and
the controller is configured to determine a state of contact with the target object based on a value detected by the contact detector.

2. The living body information measurement apparatus according to claim 1, wherein
the controller is configured to control the first light receiving member and the first light emitting member in accordance with the determined state of contact.

3. The living body information measurement apparatus according to claim 2, wherein
the contact detector includes a plurality of contact detection members,
the plurality of contact detection members each transmit a detection signal to the controller when coming into contact with the target object, and
the controller is configured to determine that the target object and the living body information detection apparatus are in contact with each other by receiving the detection signals from the plurality of contact detection members.

4. The living body information measurement apparatus according to claim 3, further comprising:
a lens member configured to transmit the first light toward the target object and transmit the first light from the target object toward the first light receiving member, and
the plurality of contact detection members are provided at the lens member.

5. The living body information measurement apparatus according to claim 4, wherein
the plurality of contact detection members are provided in a first region having a first portion that overlaps with the first light emitting member and a second region having a second portion that overlaps with the first light receiving member, and
the controller is configured to control the first light emitting member and the first light receiving member in accordance with contact states of the first region and the second region.

6. The living body information measurement apparatus according to claim 5, wherein
when at least a part of the second portion of the second region is in a non-contact state, the controller is configured to correct a light reception sensitivity at which the first light receiving member receives the first light to be higher than the light reception sensitivity at which the first light receiving member receives the first light in a case where the second portion of the second region is in a contact state.

7. The living body information measurement apparatus according to claim 6, wherein
when the controller determines that the entire second portion of the second region is in the non-contact state, the controller is configured to correct the light reception sensitivity at which the first light receiving member receives the first light to be higher than the light reception sensitivity at which the first light receiving member receives the first light in a case where the controller determines that a part of the second portion of the second region is in the non-contact state.

8. The living body information measurement apparatus according to claim 7, wherein
when the controller determines that a side of the second portion of the second region that is closer to the first portion of the first region is in the non-contact state, and a side of the second portion of the second region that is farther from the first portion of the first region is in the contact state, the controller is configured to correct the light reception sensitivity at which the first light receiving member receives the first light to be higher than the light reception sensitivity at which the first light receiving member receives the first light in a case where the controller determines that the farther side is in the non-contact state and the closer side is in the contact state.

9. The living body information measurement apparatus according to claim 8, wherein
when the controller determines that the side of the second portion of the second region that is farther from the first portion of the first region is in the non-contact state, and the side of the second portion of the second region that is closer to the first portion of the first region is in the contact state, the controller is configured not to correct the light reception sensitivity at which the first light receiving member receives the first light.

10. The living body information measurement apparatus according to claim 5, wherein
when at least a part of the first portion of the first region is in a non-contact state, the controller is configured to correct an intensity of the first light emitted by the first light emitting member to be higher than the intensity of the first light emitted by the first light emitting member in a case where the first portion of the first region is in a contact state.

11. The living body information measurement apparatus according to claim 10, wherein
when the controller determines that the entire first portion of the first region is in the non-contact state, the controller is configured to correct the intensity of the first light emitted by the first light emitting member to be higher than the intensity of the first light emitted by the first light emitting member in a case where the controller determines that a part of the first portion of the first region is in the non-contact state.

12. The living body information measurement apparatus according to claim 11, wherein
when the controller determines that a side of the first portion of the first region that is closer to the second portion of the second region is in the non-contact state, and a side of the first portion of the first region that is farther from the second portion of the second region is in the contact state, the controller is configured to correct the intensity of the first light emitted by the first light emitting member to be higher than the intensity of the first light emitted by the first light emitting member in a case where the controller determines that the farther side is in the non-contact state and the closer side is in the contact state.

13. The living body information measurement apparatus according to claim 12, wherein
when the controller determines that the side of the first portion of the first region that is farther from the second portion of the second region is in the non-contact state, and the side of the first portion of the first region that is closer to the second portion of the second region is in the contact state, the controller is configured not to correct the intensity of the first light emitted by the first light emitting member.

14. The living body information measurement apparatus according to claim 5, wherein
the controller is configured to determine a noise component contained in a signal derived from the first light received by the first light receiving member based on a change in the contact states of the first region and the second region, and acquire living body information on the target object from the signal which is derived from the first light but from which the noise component is removed.

15. The living body information measurement apparatus according to claim 5, wherein
at the lens member, the plurality of contact detection members are not provided in the first portion of the first region.

16. The living body information measurement apparatus according to claim 15, wherein
at the lens member, the plurality of contact detection members are not provided in the second portion of the second region.

17. The living body information measurement apparatus according to claim 5, further comprising:
a second light emitting member configured to emit second light having a wavelength longer than a wavelength of the first light; and a second light receiving member configured to receive the second light,
the first region of the lens member has a third portion that overlaps with the second light emitting member,
the second region of the lens member has a fourth portion that overlaps with the second light receiving member, and
the controller is configured to control the second light emitting member and the second light receiving member in accordance with contact states of the third portion and the fourth portion.

18. The living body information measurement apparatus according to claim 17, wherein
the fourth portion of the second region is farther from the first region than the second portion of the second region, and
when the first region is in a contact state and the fourth portion of the second region is in a non-contact state, the controller is configured to correct a light reception sensitivity at which the second light receiving member receives the second light in such a way that the light reception sensitivity increases, and not correct a light reception sensitivity at which the first light receiving member receives the first light.

19. The living body information measurement apparatus according to claim 18, wherein
the controller is configured to correct an intensity of the second light emitted by the second light emitting member in a case where the third portion of the first region is in the non-contact state to be higher than the intensity of the first light emitted by the first light emitting member in a case where the third portion of the first region is in the non-contact state.
